(19) United States
(12) Patent Application Publication
(10) Pub. No.: US 2026/0191605 A1
(43) Pub. Date: Jul. 9, 2026

(54) ROBOTIC SURGERY SYSTEM WITH CUSTOM HAPTIC APPROACH REGION

(71) Applicant: MAKO Surgical Corporation, Weston, FL (US)

(72) Inventors: Hans-Ulrich Becker, Mülheim (DE); Mohammad Javad Barakchi Fard, Weston, FL (US); Jason Otto, Sioux Falls, SD (US); Michael Kohnen, Weston, FL (US)

(21) Appl. No.: 19/433,410

(22) Filed: Dec. 26, 2025

Related U.S. Application Data
(60) Provisional application No. 63/743,473, filed on Jan. 9, 2025.

(51) Int. Cl. A61B 34/30; A61B 34/10; A61B 34/20

(52) U.S. Cl. CPC A61B 34/30; A61B 34/10; A61B 34/20; A61B 2034/104; A61B 2034/105; A61B 2034/107; A61B 2034/2059

(57) ABSTRACT

A method includes determining a contour of a bone model at a cross-section intersected by a haptic object corresponding to a planned resection, the haptic object including a resection region intersecting the bone model and an approach region extending away from the resection region. The method also includes morphing the approach region of the haptic object based on the contour of the bone model. The method also includes controlling a surgical robot using the morphed haptic object, wherein the surgical robot interfaces with a cutting tool that reaches the resection region via the approach region.

### CROSS-REFERENCE TOR RELATED APPLICATION

This application claims the benefit of and priority to U.S. Provisional Application No. 63/743,473 filed Jan. 9, 2025, the entire disclosure of which is incorporated by reference herein.

### BACKGROUND

The present disclosure relates generally to surgical systems for orthopedic surgeries, and more particularly to surgical systems for total and partial knee arthroplasty procedures. Knee arthroplasty, colloquially referred to as knee replacement, is widely used to treat knee osteoarthritis and other damage to a patient's knee joint by replacing portions of the knee anatomy with prosthetic components.

One possible tool for use in total knee arthroplasty procedure is a robotically-assisted surgical system. A robotically-assisted surgical system typically includes a robotic device that is used to prepare a patient's anatomy, a tracking system configured to monitor the location of the robotic device relative to the patient's anatomy, and a computing system configured to monitor and control the robotic device. Robotically-assisted surgical systems, in various forms, autonomously carry out surgical tasks, provide force feedback to a user manipulating a surgical device to complete surgical tasks, augment surgeon dexterity and precision, and/or provide other navigational cues to facilitate safe and accurate surgical operations.

A surgical plan is typically established prior to performing a surgical procedure with a robotically-assisted surgical system. Based on the surgical plan, the surgical system guides, controls, or limits movements of the surgical tool during portions of the surgical procedure. Guidance and/or control of the surgical tool serves to protect the patient and to assist the surgeon during implementation of the surgical plan.

### SUMMARY

One implementation of the present disclosure is a method. The method includes determining a contour of a bone model at a cross-section intersected by a haptic object corresponding to a planned resection, the haptic object comprising a resection region intersecting the bone model and an approach region extending away from the resection region. The method also includes morphing the approach region of the haptic object based on the contour of the bone model. The method also includes controlling a surgical robot using the morphed haptic object, wherein the surgical robot interfaces with a cutting tool that reaches the resection region via the approach region.

Another implementation of the present disclosure is a system. The system includes a robotic device and a circuitry. The circuitry is configured to determine a contour of a bone model at a cross-section intersected by a haptic object corresponding to a planned resection, the haptic object comprising a resection region intersecting the bone model and an approach region extending away from the resection region. The circuitry is also configured to morph the approach region of the haptic object based on the contour of the bone model. The circuitry is also configured to control the robotic device using the morphed haptic object, wherein the surgical robot interfaces with a cutting tool that reaches the resection region via the approach region.

Another implementation of the present disclosure relates to one or more non-transitory computer-readable media storing instructions that, when executed by a processor, cause the processor to perform operations. The operations include determining a contour of a bone model at a cross-section intersected by a haptic object corresponding to a planned resection, the haptic object comprising a resection region intersecting the bone model and an approach region extending away from the resection region. The operations also include morphing the approach region of the haptic object based on the contour of the bone model. The operations also include controlling a surgical robot using the morphed haptic object, wherein the surgical robot interfaces with a cutting tool that reaches the resection region via the approach region.

### BRIEF DESCRIPTION OF THE DRAWINGS

FIG. 1 is a perspective view of a post-operative prosthetic knee joint fitted with a prosthetic system.

FIG. 2 is an illustration of a surgical system, according to an exemplary embodiment.

FIG. 3 is a schematic diagram of a computer system that may be associated with the surgical system of FIG. 2, according to an exemplary embodiment.

FIG. 4 is an illustration of a graphical user interface of the surgical system of FIG. 2, according to an exemplary embodiment.

FIG. 5 is a first illustration of a standard haptic boundary, according to an exemplary embodiment.

FIG. 6 is a second first illustration of the standard haptic boundary, according to an exemplary embodiment.

FIG. 7 is an illustration of a customized haptic boundary, according to an exemplary embodiment.

FIG. 8 is a first flowchart of a process for generating customized haptic boundaries based on patient-specific anatomic data, according to an exemplary embodiment.

FIG. 9 is a first illustration of a step from the process of FIG. 8, according to an exemplary embodiment.

FIG. 10 is a second illustration of a step from the process of FIG. 8, according to an exemplary embodiment.

FIG. 11 is a third illustration of a step from the process of FIG. 8, according to an exemplary embodiment.

FIG. 12 is a fourth illustration of a step from the process of FIG. 8, according to an exemplary embodiment.

FIG. 13 is a fifth illustration of a step from the process of FIG. 8, according to an exemplary embodiment.

FIG. 14 is a flowchart of a process for generating a customized haptic approach region during the process of FIG. 8, according to an exemplary embodiment.

FIG. 15 is a flowchart of a process for calculating the customized haptic approach region during the process of FIG. 14, according to an exemplary embodiment.

FIG. 16A is a first illustration of a step from the process of FIG. 15, according to an exemplary embodiment.

FIG. 16B is a second illustration of a step from the process of FIG. 15, according to an exemplary embodiment.

FIG. 16C is a third illustration of a step from the process of FIG. 15, according to an exemplary embodiment.

FIG. 17 is a second flowchart of a process for generating a customized haptic boundary based on patient-specific anatomic data, according to an exemplary embodiment.

### DETAILED DESCRIPTION

Presently preferred embodiments of the invention are illustrated in the drawings. An effort has been made to use the same or like reference numbers throughout the drawings to refer to the same or like parts. Although this specification refers primarily to a robotic arm for orthopedic knee replacement, it should be understood that the subject matter described herein is applicable to other types of robotic systems, including those used for surgical and non-surgical applications, as well as to other joints of the body, such as, for example, a hip or shoulder joint.

Existing haptic boundaries applied during surgical procedures, such as a total knee arthroplasty (“TKA”), may limit injury to surrounding tissues or other anatomical features that are not intended to be treated during the surgical procedure. Such existing haptic boundaries, however, may not account for an approach region by which a surgical tool being used to perform the surgical procedure accesses a resection region. Therefore, although existing haptic boundaries may protect anatomical features surrounding the resection region such that the surgical tool does not inadvertently damage healthy tissue or other features during resection, such haptic boundaries may not protect the patient's anatomy surrounding a region by which the surgical tool approached the resection region. In the case of a TKA, for instance, the anterior cruciate ligament (ACL) and/or the posterior cruciate ligament (PCL) may risk being damaged while the surgical tool approaches a resection region of the tibia.

The systems and methods described herein, however, propose generating a customized approach region for a haptic object based on patient-specific anatomy and implant-specific parameters. Therefore, the systems and methods described herein provide a solution for enhancing technologies used to perform robotically-assisted surgeries with haptic object. For example, in a “cruciate-retaining” application such as a TKA, the prosthetic implant components may be configured to avoid interference with or impingement on the retained cruciate ligaments passing through the intercondylar area of the knee joint. Limiting the amount of disturbance of native tissue at the attachment sites helps preserve the natural anchoring mechanism of the tissue, which decreases the likelihood of failure at the attachment site. As such, customizing the approach region of the haptic object by which a surgical tool reaches the attachment site further decreases the likelihood of failure at the attachment site by protecting native tissue such as the ACL and/or the PCL.

A healthy knee joint comprises the interface between the distal end of the femur and the proximal end of the tibia. If the healthy knee joint becomes damaged due, for example, to injury or disease, knee surgery may be required to restore normal structure and function of the joint. If the damage to the knee is severe, TKA may be required. TKA typically involves the removal of the damaged portion of joint and the replacement of the damaged portion of the joint with one or more prosthetic components.

In some TKA procedures, one or more of cruciate ligaments (including anterior cruciate ligament and/or posterior cruciate ligament) may be left intact, to be re-used with the prosthetic implants to form the new knee joint. In these “cruciate-retaining” applications, the prosthetic implant components may be configured to avoid interference with or impingement on the retained cruciate ligaments passing through the intercondylar area of the knee joint. For example, each of the femoral and tibial prosthetic components may be designed with a intercondylar “notch” that extends from the posterior of the prosthetic component toward the anterior of the prosthetic component. The femoral and tibial intercondylar notches provide a passage that allows the cruciate ligament to pass from the femoral intercondylar fossa down to the tibial eminence.

Because cruciate ligaments are exposed to significant tensile force during normal knee joint use, it is important that the attachment sites where the cruciate ligaments attach to the femur and tibia have sufficient strength to properly anchor the cruciate ligaments to the bone. Otherwise, the force applied by the cruciate ligament strains the tissue around the attachment site, possibly leading to failure of the joint, which may require corrective surgery to repair. One way to limit the possibility of such a failure is to limit the amount of bone resected at or near the attachment site(s) (i.e., the intercondylar fossa of the femur and the tibial eminence of the tibia). Limiting the amount of disturbance of native tissue at the attachment sites helps preserve the natural anchoring mechanism of the tissue, which decreases the likelihood of failure at the attachment site.

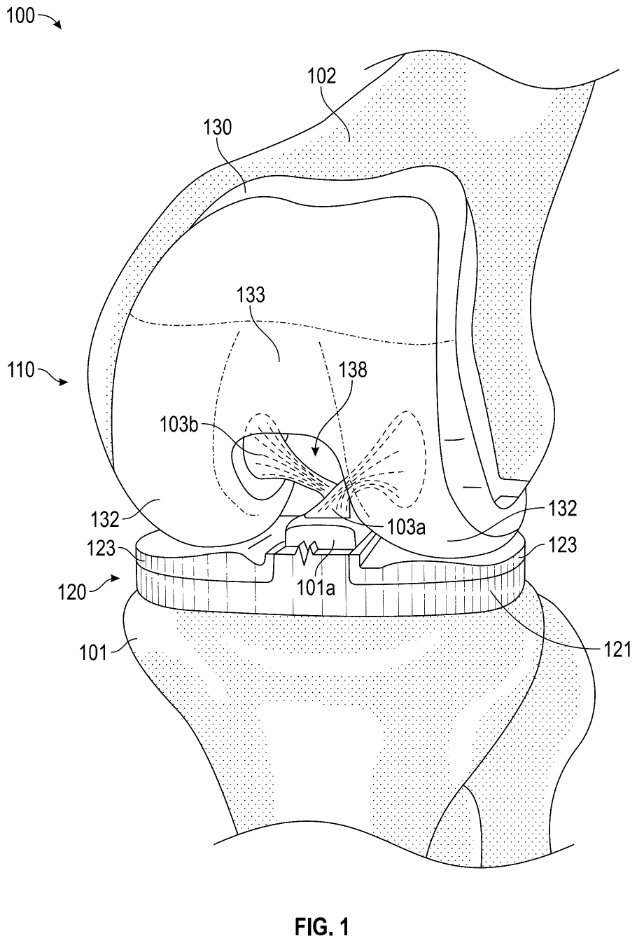

In the embodiment illustrated in FIG. 1, a knee joint 100 is shown with a prosthetic implant system 110 including a number of components configured to replace a resected portion of a native knee joint. According to one embodiment, the prosthetic implant system 110 includes a tibial implant system 120 configured to replace a resected portion of a native tibia 101. The prosthetic implant system 110 also includes a femoral component 130 configured to replace a resected portion of a native femur 102. After implantation during knee replacement surgery, the tibial implant system 120 and the femoral component 130 cooperate to replicate the form and function of the native knee joint.

The femoral component 130 is secured to a distal end of the native femur 102 and configured to replace the structure and function of a native femoral portion of the knee joint 100 (e.g., the native femur 102). As such, the femoral component 130 may be manufactured from surgical-grade metal or metal alloy material (such as surgical-grade steel, titanium or titanium alloy, a cobalt-chromium alloy, a zirconium alloy, or tantalum) that is substantially rigid for providing sufficient strength to support the forces required of the knee joint. According to one embodiment, the femoral component 130 may embody a single component having a plurality of different structural features, each configured to perform a particular function associated with the knee joint 100. For example, the femoral component 130 may include a pair of condyles 132, each of which is coupled to a patellar guide portion 133. The pair of condyles 132 are separated from one another by an intercondylar notch 138, which provides a channel through which one or more cruciate ligaments, such as anterior cruciate ligament (ACL) 103a and/or posterior cruciate ligament (PCL) 103b, may pass.

The tibial implant system 120 may include a plurality of components that cooperate to provide a stable surface that articulates with the femoral component 130 to restore proper knee joint function. As illustrated in FIG. 1, the tibial implant system 120 includes a base portion 121 and one or more insert portions 123. During a knee replacement procedure, the base portion 121 is secured to a proximal end of the native tibia 101, which has been surgically prepared by removing damaged bone and tissue and reshaping the healthy bone to receive the base portion 121. Once the base portion 121 is secured to the native tibia 101, the surgeon completes assembly of the tibial implant system 120 by engaging and securing the one or more insert portions 123 within the base portion 121. The base portion 121 of the tibial implant system 120 may be configured with a passage through the center to allow for connection between the retained cruciate ligaments (e.g., ACL 103a, PCL 103b) and a tibial eminence 101a.

The base portion 121 may be configured to emulate the structure and function of a top surface of the native tibia 101. Thus, similar to the femoral component 130, the base portion 121 may be manufactured from surgical-grade metal or metal alloy material (such as surgical-grade steel, titanium or titanium alloy, a cobalt-chromium alloy, a zirconium alloy, or tantalum) that is substantially rigid for providing a stable base upon which to reconstruct the remainder of the prosthetic joint.

The one or more insert portions 123 may be designed to emulate the form and function of certain components of the natural femorotibial interface, including, among other things, medial and lateral menisci of the knee joint 100. As such, the one or more insert portions 123 may be constructed of smooth, semi-rigid synthetic or semi-synthetic plastic, rubber, or polymer material. The one or more insert portions 123 may be configured to provide a smooth surface that is designed to articulate with the femoral component 130 during a normal knee operation. According to one embodiment, the one or more insert portions 123 are configured to removably engage with the base portion 121. Accordingly, the one or more insert portions 123 are configured for periodic replacement if the one or more insert portions 123 deteriorate over time due to, for example, excessive wear.

In order to ensure precise and accurate preparation of the joint to receive a prosthetic implant, a computer-assisted surgical system may be used to generate a graphical representation of the surgical site and a corresponding virtual guide that may aid the surgeon in properly aligning the tool prior to interaction with a patient's anatomy. Many computer-assisted surgical systems include software that allows users to electronically register certain anatomic features (e.g., bones, soft tissues, etc.), surgical instruments, and other landmarks associated with the surgical site. Therefore, computer-assisted surgical systems may generate a graphical representation of the surgical site based on the registration of the anatomic features. The software of such computer-assisted surgical systems also allows users to plan certain aspects of the surgical procedure and register these aspects for display with the graphical representation of the surgical site. For example, in a knee joint replacement procedure, a surgeon may register target navigation points, the location and depth of bone and tissue cuts, virtual boundaries that may be associated with a corresponding reference for the application of haptic force, and other aspects of the surgery.

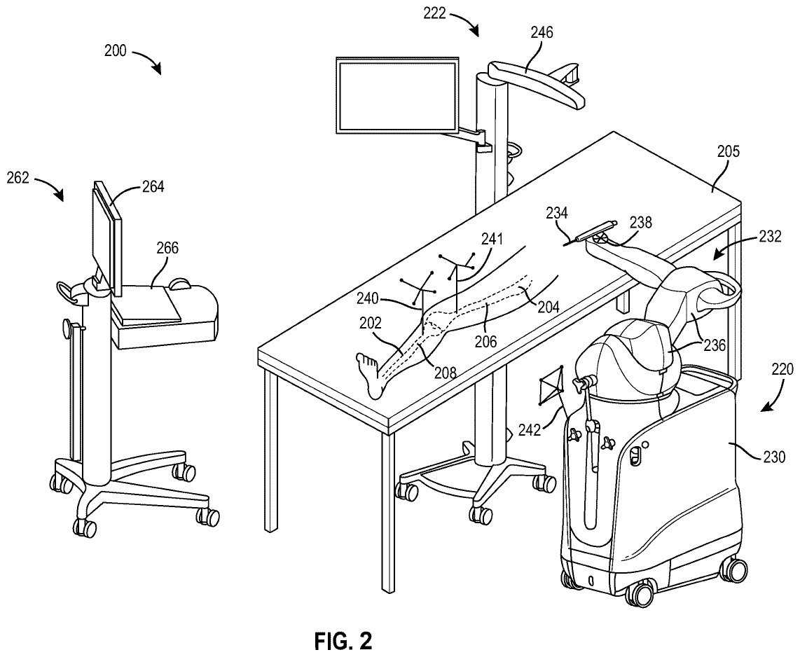

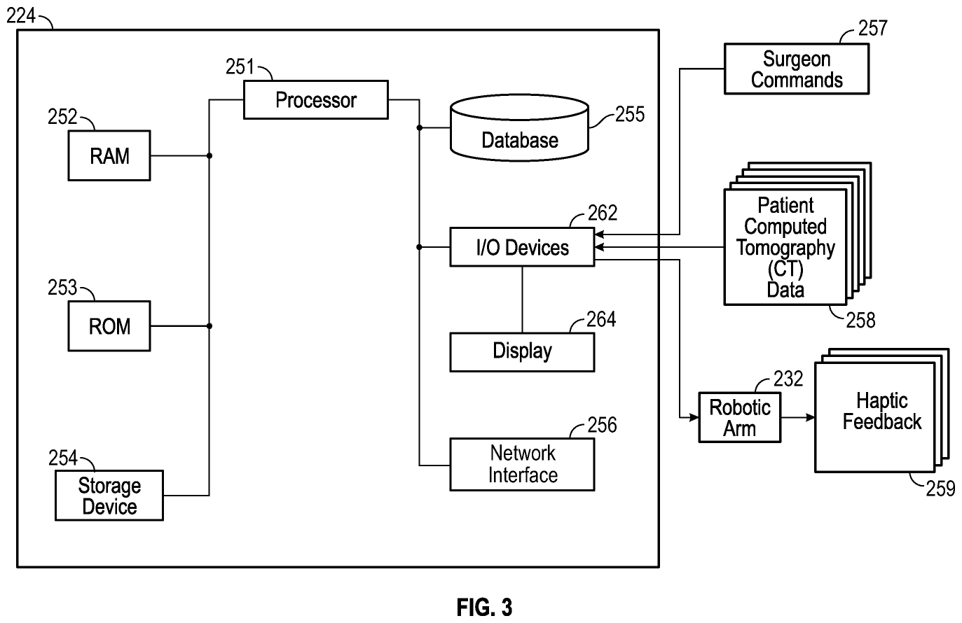

Referring now to FIGS. 2 and 3, a surgical system 200 for orthopedic surgery is shown, according to an exemplary embodiment. In general, the surgical system 200 is configured to facilitate the planning and execution of a surgical plan, for example to facilitate a joint-related procedure. As shown in FIG. 2, the surgical system 200 is set up to treat a leg 202 of a patient 204 sitting or lying on table 205. In the illustration shown in FIG. 2, the leg 202 includes femur 206 (which may include the native femur 102) and tibia 208 (e.g., which may include the native tibia 101), between which a prosthetic knee implant (e.g., the prosthetic implant system 110, as described above and shown in FIG. 1) is to be implanted in a TKA procedure. In other scenarios, the surgical system 200 is set up to treat a hip joint of a patient, i.e., a femur and a pelvis of the patient. Additionally, in still other scenarios, the surgical system 200 is set up to treat a shoulder of a patient, i.e., to facilitate replacement and/or augmentation of components of a shoulder joint (e.g., to facilitate placement of a humeral component, a glenoid component, and a graft or implant augment). Various other anatomical regions and procedures are also possible. To facilitate the procedure, surgical system 200 includes robotic device 220, tracking system 222, and computing system 224.

The robotic device 220 is configured to modify a patient's anatomy (e.g., femur 206 of patient 204) under the control of the computing system 224. One embodiment of the robotic device 220 is a haptic device. “Haptic” refers to a sense of touch, and the field of haptics relates to, among other things, human interactive devices that provide feedback (e.g., haptic feedback 259) to an operator. Feedback may include tactile sensations such as, for example, vibration. Feedback may also include providing force to a user, such as a positive force or a resistance to movement. One use of haptics is to provide a user of the device with guidance or limits for manipulation of that device. For example, a haptic device may be coupled to a surgical tool (e.g., surgical tool 234), which can be manipulated by a surgeon to perform a surgical procedure. The surgeon's manipulation of the surgical tool can be guided or limited through the use of haptics to provide feedback to the surgeon during manipulation of the surgical tool, as described in greater detail herein.

Another embodiment of the robotic device 220 is an autonomous or semi-autonomous robot. “Autonomous” refers to a robotic device's ability to act independently or semi-independently of human control by gathering information about its situation, determining a course of action, and automatically carrying out that course of action. For example, in such an embodiment, the robotic device 220, in communication with the tracking system 222 and the computing system 224, may autonomously complete a series of femoral cuts without direct human intervention.

A robotic arm 232 is configured to support the surgical tool 234 and provide a force as instructed by the computing system 224. In some embodiments, the robotic arm 232 allows a user to manipulate the surgical tool 234 and provides force feedback to the user (e.g., haptic feedback 259). In such an embodiment, the robotic arm 232 includes joints 236 and mount 238 that include motors, actuators, or other mechanisms configured to allow a user to freely translate and rotate the robotic arm 232 and the surgical tool 234 through allowable poses while providing force feedback to constrain or prevent some movements of the robotic arm 232 and the surgical tool 234 as instructed by computing system 224. As described in detail below, the robotic arm 232 thereby allows a surgeon to have full control over the surgical tool 234 within a control object while providing force feedback along a boundary of that object (e.g., a vibration, a force preventing or resisting penetration of the boundary). In some embodiments, the robotic arm 232 is configured to move the surgical tool 234 to a new pose automatically without direct user manipulation, as instructed by computing system 224, in order to position the robotic arm 232 as needed and/or complete certain surgical tasks, including, for example, cuts in the femur 206 and/or the tibia 208.

The surgical tool 234 is configured to cut, burr, grind, drill, partially resect, reshape, and/or otherwise modify a bone. The surgical tool 234 may be any suitable tool, and may be one of multiple tools interchangeably connectable to robotic device 220. For example, as shown in FIG. 2 the surgical tool 234 is a sagittal saw with a blade aligned parallel with a tool axis or perpendicular to the tool axis. As another example, the surgical tool 234 may be a spherical burr. The surgical tool 234 may also be a holding arm or other support configured to hold an implant component in position while the implant component is screwed to a bone, adhered (e.g., cemented) to a bone or other implant component, or otherwise installed in a preferred position. In some embodiments, the surgical tool 234 is an impaction tool configured to provide an impaction force to an implant to facilitate fixation of the implant to a bone in a planned location and orientation.

The tracking system 222 is configured to track the patient's anatomy (e.g., the femur 206 and the tibia 208) and the robotic device 220 (i.e., the surgical tool 234 and/or the robotic arm 232) to enable control of the surgical tool 234 coupled to the robotic arm 232, to determine a position and orientation of modifications or other results made by the surgical tool 234, and allow a user to visualize the bones (e.g., the femur 206, the tibia 208, a pelvis, a humerus, a scapula, etc., as applicable in various procedures), the surgical tool 234, and/or the robotic arm 232 on a display (e.g., display 264) of the computing system 224. More particularly, the tracking system 222 determines a position and orientation (i.e., pose) of objects (e.g., the surgical tool 234, the femur 206, the tibia 208) with respect to a coordinate frame of reference and tracks (i.e., continuously determines) the pose of the objects during a surgical procedure. According to various embodiments, the tracking system 222 may be any type of navigation system, including a non-mechanical tracking system (e.g., an optical tracking system), a mechanical tracking system (e.g., tracking based on measuring the relative angles of the joints 236 of the robotic arm 232), or any combination of non-mechanical and mechanical tracking systems.

In the embodiment shown in FIG. 2, the tracking system 222 includes an optical tracking system. Accordingly, the tracking system 222 includes a first fiducial tree 240 coupled to the tibia 208, a second fiducial tree 241 coupled to the femur 206, a third fiducial tree 242 coupled to a base 230 of the robotic device 220, one or more fiducials coupled to surgical tool 234, and a detection device 246 configured to detect the three-dimensional position of fiducials (i.e., markers on fiducial trees 240-242). The fiducial trees 240, 241 may be coupled to other bones as suitable for various procedures (e.g., a pelvis and a femur in a hip arthroplasty procedure). The detection device 246 may be an optical detector such as a camera or infrared sensor. The fiducial trees 240-242 include fiducials, which are markers configured to show up clearly to the optical detector and/or be easily detectable by an image processing system using data from the optical detector, for example by being highly reflective of infrared radiation (e.g., emitted by an element of the tracking system 222). A stereoscopic arrangement of cameras on the detection device 246 allows the position of each fiducial to be determined in 3D-space through a triangulation approach. Each fiducial has a geometric relationship to a corresponding object, such that tracking of the fiducials allows for the tracking of the object (e.g., tracking the second fiducial tree 241 allows the tracking system 222 to track the femur 206), and the tracking system 222 may be configured to carry out a registration process to determine or verify this geometric relationship. Unique arrangements of the fiducials in the fiducial trees 240-242 (i.e., the fiducials in the first fiducial tree 240 are arranged in a different geometry than fiducials in the second fiducial tree 241) allows for distinguishing the fiducial trees 240-242, and therefore the objects being tracked, from one another.

Using the tracking system 222 of FIG. 2 or some other approach to surgical navigation and tracking, the surgical system 200 can determine the position of the surgical tool 234 relative to a patient's anatomical feature, for example the femur 206, as the surgical tool 234 is used to modify the anatomical feature or otherwise facilitate the surgical procedure. Additionally, using the tracking system 222 of FIG. 2 or some other approach to surgical navigation and tracking, the surgical system 200 can determine the relative poses of the tracked bones.

The computing system 224 is configured to create a surgical plan, control the robotic device 220 in accordance with the surgical plan to make one or more bone modifications and/or facilitate implantation of one or more prosthetic components. Accordingly, the computing system 224 is communicably coupled to the tracking system 222 and the robotic device 220 to facilitate electronic communication between the robotic device 220, the tracking system 222, and the computing system 224. Further, the computing system 224 may be connected to a network to receive information related to a patient's medical history or other patient profile information, medical imaging, surgical plans, surgical procedures, and to perform various functions related to performance of surgical procedures, for example by accessing an electronic health records system. As shown in FIG. 3, the patient profile information may include patient computed-tomography (CT) data 258.

As illustrated in FIG. 3, the computing system 224 may include one or more hardware and/or software components configured to execute software programs, such as, tracking software, surgical navigation software, 3-D bone modeling or imaging software, and/or software for establishing and modifying virtual haptic boundaries for use with a force system to provide haptic feedback to the surgical tool 234. The computing system 224 may include one or more hardware components such as, for example, a central processing unit (CPU), shown as processor 251; a memory device, such as a random-access memory (RAM) module 252, a read-only memory (ROM) module 253, and/or a storage device 254; a database 255; one or more input/output (I/0) devices 262; and a network interface 256. The computing system 224 may include additional, fewer, and/or different components than those listed above. It is understood that the components listed above are exemplary only and not intended to be limiting.

The processor 251 may include one or more microprocessors, each configured to execute instructions and process data to perform one or more functions associated with the computing system 224. The processor 251 can be implemented as a general-purpose processor, an application specific integrated circuit (ASIC), one or more field programmable gate arrays (FPGAs), a group of processing components, or other suitable electronic processing components. As illustrated in FIG. 3, the processor 251 may be communicatively coupled to the RAM 252, the ROM 253, the storage device 254, the database 255, the I/O devices 262, and the network interface 256. The processor 251 may be configured to execute sequences of computer program instructions to perform various processes, which will be described in detail below. In some embodiments, the computer program instructions may be loaded into the RAM 252 for execution by the processor 251.

The memory device (e.g., memory, memory unit, storage device, etc.) is one or more devices (e.g., RAM, ROM, Flash memory, hard disk storage, etc.) for storing data and/or computer code for completing or facilitating the various processes and functions described in the present application. In some embodiments, the memory device may be or include volatile memory or non-volatile memory. The memory device may include database components, object code components, script components, or any other type of information structure for supporting the various activities and information structures described in the present application. That is, the memory device (e.g., memory, memory unit, storage device, etc.), such as the RAM 252, the ROM 253, and/or the storage device 254, may be configured to store computer-readable instructions that, when executed by the processor 251, may cause the surgical system 200 or one or more constituent components, such as the tracking system 222, to perform functions or tasks associated with surgical system 200. For example, the memory device may include instructions for causing the surgical system 200 to perform one or more methods for determining changes in parameters of the knee joint 100 after a TKA procedure. The memory device may also contain instructions that cause the surgical system 200 to capture positions of a plurality of anatomic landmarks associated with certain registered objects, such as the surgical tool 234 or portions of a patient's anatomy, and cause the computing system 224 to generate virtual representations of the registered objects for display on the I/O devices 262.

The I/O devices 262 may include one or more components configured to communicate information with a user associated with the surgical system 200. That is, the I/O devices 262 are configured to receive user input and display output as needed for the functions and processes described herein. As shown in FIGS. 2 and 3, the I/O devices 262 includes a display 264 and a keyboard 266. The display 264 is configured to display graphical user interfaces generated by the computing system 224 that include, for example, information about surgical plans, medical imaging, settings and other options for the surgical system 200, status information relating to the tracking system 222 and the robotic device 220, and tracking visualizations based on data supplied by the tracking system 222. The keyboard 266 is configured to receive user input to those graphical user interfaces to control one or more functions of the surgical system 200. For example, the I/O devices 262 may include a console with the keyboard 266 and a mouse, the console allowing a user (e.g., a surgeon) to input parameters (e.g., surgeon commands 250) associated with the surgical system 200.

In some embodiments, the I/O devices 262 may also include peripheral devices such as, for example, a printer for printing information associated with the surgical system 200, a user-accessible disk drive (e.g., a USB port, a floppy, CD-ROM, or DVD-ROM drive, etc.) to allow a user to input data stored on a portable media device, a microphone, a speaker system, or any other suitable type of interface device. For example, the I/O devices 262 may include an electronic interface that allows a user to input patient computed tomography (CT) data 258 into the surgical system 200. The CT data 258 may then be used to generate and manipulate virtual representations of portions of the patient's anatomy (e.g., a virtual model of the tibia 208).

In some embodiments, the computing system 224 is configured to facilitate the creation of a preoperative surgical plan prior to the surgical procedure. According to some embodiments, the preoperative surgical plan is developed utilizing a three-dimensional representation of a patient's anatomy, also referred to herein as a “virtual bone model.” A “virtual bone model” may include virtual representations of cartilage or other tissue in addition to bone. To obtain the virtual bone model, the computing system 224 receives imaging data, such as the patient CT data 258, of the patient's anatomy on which the surgical procedure is to be performed (e.g., femur 206, tibia 208). The imaging data may be created using any suitable medical imaging technique to image the relevant anatomical feature, including computed tomography (CT), magnetic resonance imaging (MRI), and/or ultrasound. The imaging data is then segmented (i.e., the regions in the imaging data corresponding to different anatomical features are distinguished) to obtain the virtual bone model.

Alternatively, the virtual bone model may be obtained by selecting a three-dimensional model from a database or library of bone models, such as the database 255. In one embodiment, the user may use the I/O devices 262 to select an appropriate model. In another embodiment, the computing system 224 may execute stored instructions to select an appropriate model from the database 255 based on images or other information provided about the patient. The selected bone model(s) from the database can then be deformed based on specific patient characteristics, creating a virtual bone model for use in surgical planning and implementation as described herein.

A preoperative surgical plan can then be created based on the virtual bone model. The surgical plan may be automatically generated by the computing system 224, input by a user via the I/O devices 262, or some combination of the two (e.g., computing system 224 limits some features of user-created plans, generates a plan that a user can modify, etc.). In some embodiments, as described in detail below, the surgical plan may be generated and/or modified based on distraction force measurements collected intraoperatively. In some embodiments, the surgical plan may be modified based on qualitative intra-operational assessment of implant fixation (i.e., loose or fixed) and/or intra-operative bone defect mapping after an existing (i.e., primary, etc.) implant removal.

The preoperative surgical plan includes the desired cuts, holes, surfaces, burrs, or other modifications to a patient's anatomy to be made using the surgical system 200. For example, for a TKA procedure, the preoperative plan may include the cuts necessary to form, on the femur 206, a distal surface, a posterior chamfer surface, a posterior surface, an anterior surface, and an anterior chamfer, surfaces in relative orientations and positions suitable to be mated to corresponding surfaces of a prosthetic to be joined to the femur (e.g., the femoral component 130 of the prosthetic implant system 110) during the surgical procedure, as well as cuts necessary to form, on the tibia 208, surface(s) suitable to mate to the prosthetic to be joined to the tibia (e.g., the tibial implant system 120) during the surgical procedure. As another example, in a hip arthroplasty procedure, the surgical plan may include the burr necessary to form one or more surfaces on the acetabular region of the pelvis to receive a cup and, in suitable cases, an implant augment. Accordingly, the computing system 224 may receive, access, and/or store a model of the prosthetic to facilitate the generation of surgical plans.

The computing system 224 is further configured to generate a control object for the robotic device 220 in accordance with the surgical plan. The control object may take various forms according to the various types of possible robotic devices (e.g., haptic, autonomous, etc.). For example, in some embodiments, the control object defines instructions for the robotic device 220 to control the robotic arm 232 to move within the control object (i.e., to autonomously make one or more cuts of the surgical plan guided by feedback from the tracking system 222). In some embodiments, the control object includes a visualization of the surgical plan and the robotic device 220 on the display 264 to facilitate surgical navigation and help guide a surgeon to follow the surgical plan (e.g., without active control or force feedback of the robotic device 220). In embodiments where the robotic device 220 is a haptic device, the control object may be a haptic object as described in the following paragraphs.

Where the robotic device 220 is a haptic device, the computing system 224 is further configured to generate one or more haptic objects based on the preoperative surgical plan to assist the surgeon during implementation of the surgical plan by enabling constraint of the surgical tool 234 during the surgical procedure. A haptic object may be formed in one, two, or three dimensions. For example, a haptic object can be a line, a plane, or a three-dimensional volume. A haptic object may be curved with curved surfaces and/or have flat surfaces, and can be any shape, for example a funnel shape. Haptic objects can be created to represent a variety of desired outcomes for movement of the surgical tool 234 during the surgical procedure. One or more of the boundaries of a three-dimensional haptic object may represent one or more modifications, such as cuts, to be created on the surface of a bone. A planar haptic object may represent a modification, such as a cut, to be created on the surface of a bone. A curved haptic object may represent a resulting surface of a bone as modified to receive an implant and/or an implant augment.

In an embodiment where the robotic device 220 is a haptic device, the computing system 224 is further configured to generate a virtual tool representation of the surgical tool 234. The virtual tool includes one or more haptic interaction points (HIPs), which represent and are associated with locations on the physical surgical tool 234. For example, in an embodiment in which the surgical tool 234 is a spherical burr, a HIP may represent the center of the spherical burr. If the surgical tool 234 is an irregular shape, for example as for a sagittal saw (e.g., as shown in FIG. 2), the virtual representation of the sagittal saw may include numerous HIPs. Using multiple HIPs to generate haptic forces (e.g. positive force feedback or resistance to movement) on a surgical tool is described in U.S. application Ser. No. 13/339,369, titled “System and Method for Providing Substantially Stable Haptics,” filed Dec. 28, 2011, and hereby incorporated by reference herein in its entirety. In one embodiment of the present disclosure, a virtual tool representing a sagittal saw includes eleven HIPs. As used herein, references to a “HIP” are deemed to also include references to “one or more HIPs.” As described below, relationships between HIPs and haptic objects enable the surgical system 200 to constrain the surgical tool 234.

Prior to performance of the surgical procedure, the patient's anatomy (e.g., the femur 206, the tibia 208) is registered to the virtual bone model of the patient's anatomy by any known registration technique. One possible registration technique is point-based registration, as described in U.S. Pat. No. 8,010,180, titled “Haptic Guidance System and Method,” granted Aug. 30, 2011, and hereby incorporated by reference herein in its entirety. Alternatively, registration may be accomplished by 2D/3D registration utilizing a hand-held radiographic imaging device, as described in U.S. application Ser. No. 13/562,163, titled “Radiographic Imaging Device,” filed Jul. 30, 2012, and hereby incorporated by reference herein in its entirety. Registration also includes registration of the surgical tool 234 to a virtual tool representation of the surgical tool 234, so that the surgical system 200 can determine and monitor the pose of the surgical tool 234 relative to the patient (i.e., to the femur 206, the tibia 208). Registration allows for accurate navigation, control, and/or force feedback during the surgical procedure.

The computing system 224 is configured to monitor the virtual positions of the virtual tool representation, the virtual bone model, and the control object (e.g., virtual haptic objects) corresponding to the real-world positions of the patient's bone (e.g., the femur 206, the tibia 208), the surgical tool 234, and one or more lines, planes, or three-dimensional spaces defined by forces created by robotic device 220. For example, if the patient's anatomy moves during the surgical procedure as tracked by the tracking system 222, the computing system 224 correspondingly moves the virtual bone model. The virtual bone model therefore corresponds to, or is associated with, the patient's actual (i.e. physical) anatomy and the position and orientation of that anatomy in real/physical space. Similarly, any haptic objects, control objects, or other planned automated robotic device motions created during surgical planning that are linked to cuts, modifications, etc. to be made to that anatomy also move in correspondence with the patient's anatomy. In some embodiments, the surgical system 200 includes a clamp or brace to substantially immobilize the patient's bone to minimize the need to track and process motion of the patient's bone.

For embodiments where the robotic device 220 is a haptic device, the surgical system 200 is configured to constrain the surgical tool 234 based on relationships between HIPs and haptic objects. That is, when the computing system 224 uses data supplied by the tracking system 222 to detect that a user is manipulating the surgical tool 234 to bring a HIP in virtual contact with a haptic object, the computing system 224 generates a control signal to the robotic arm 232 to provide the haptic feedback 259 (e.g., a force, a vibration) to the user to communicate a constraint on the movement of the surgical tool 234. In general, the term “constrain,” as used herein, is used to describe a tendency to restrict movement. However, the form of constraint imposed on the surgical tool 234 depends on the form of the relevant haptic object. A haptic object may be formed in any desirable shape or configuration. As noted above, three exemplary embodiments include a line, plane, or three-dimensional volume. In one embodiment, the surgical tool 234 is constrained because a HIP of surgical tool 234 is restricted to movement along a linear haptic object. In another embodiment, the haptic object is a three-dimensional volume and the surgical tool 234 may be constrained by substantially preventing movement of the HIP outside of the volume enclosed by the walls of the three-dimensional haptic object. In another embodiment, and as described in greater detail herein, the surgical tool 234 is constrained because a planar haptic object substantially prevents movement of the HIP outside of the plane and outside of the boundaries of the planar haptic object. For example, the computing system 224 can establish a planar haptic object corresponding to a planned planar distal cut needed to create a distal surface on the femur 206 in order to confine the surgical tool 234 substantially to the plane needed to carry out the planned distal cut.

For embodiments where the robotic device 220 is an autonomous device, the surgical system 200 is configured to autonomously move and operate the surgical tool 234 in accordance with the control object. For example, the control object may define areas relative to the femur 206 for which a cut should be made. In such a case, one or more motors, actuators, and/or other mechanisms of the robotic arm 232 and the surgical tool 234 are controllable to cause the surgical tool 234 to move and operate as necessary within the control object to make a planned cut, for example using tracking data from the tracking system 222 to allow for closed-loop control.

Systems and methods consistent with the disclosed embodiments provide a solution for customizing a virtual haptic boundary and providing haptic feedback for guiding the surgical instrument. According to one embodiment, the virtual haptic boundary may be customized based on a user request to modify a default boundary associated with a corresponding implant geometry. Alternatively or additionally, the virtual haptic boundary may be customized based, at least in part, on a detection of the patient's anatomy (e.g., a location of soft tissue, the edge perimeter of a bone, etc.). The process for customizing the virtual haptic boundary may be part of an implant planning phase, during which the surgeon pre-operatively or intra-operatively plans the placement of prosthetic implants and the corresponding modification/removal of joint tissue to accommodate the implant.

Systems and methods consistent with the disclosed embodiments provide a solution for customizing a virtual haptic boundary and providing haptic feedback 259 for guiding the surgical tool 234. According to one embodiment, the virtual haptic boundary may be customized based on a user request to modify a default boundary associated with a corresponding implant geometry (e.g., the prosthetic implant system 110). Alternatively or additionally, the virtual haptic boundary may be customized based on a detection of the patient's anatomy (e.g., a location of soft tissue, the edge perimeter of a bone, etc.). More specifically, the virtual haptic boundary may be customized based, at least in part, on an approach region that is unique to the patient's anatomy (e.g., such that as the surgical tool 234 approaches a resection region, the surgical tool 234 avoids interference with surrounding anatomical structures). The process for customizing the virtual haptic boundary may be part of an implant planning phase, during which the surgeon pre-operatively or intra-operatively plans the placement of prosthetic implants and the corresponding modification/removal of joint tissue to accommodate the implant.

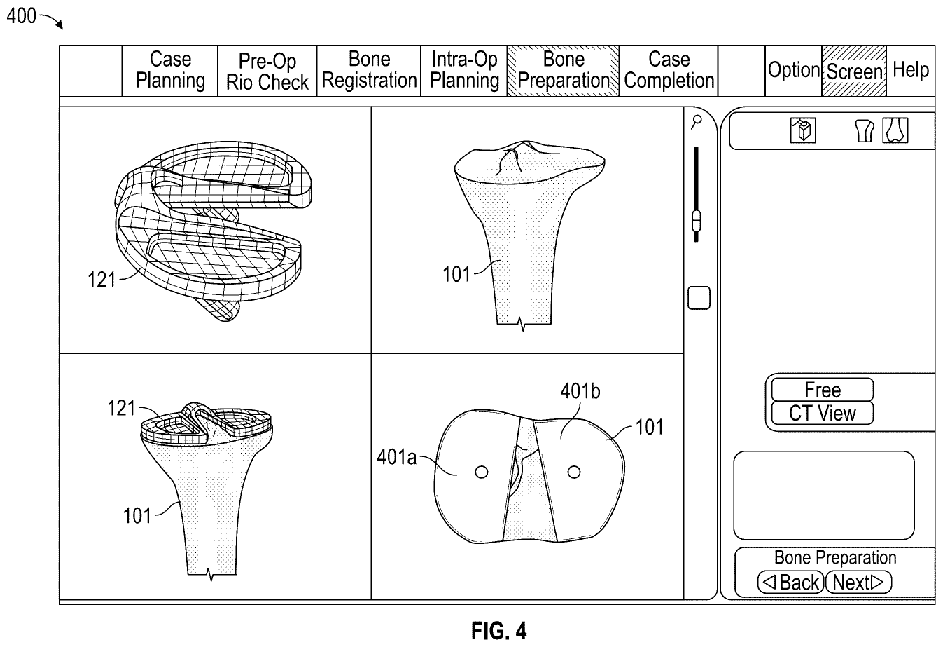

Referring now to FIG. 4, a graphical user interface 400 generated the computing system 224 is shown. As illustrated in FIG. 4, the graphical user interface 400 may include virtual models of prosthetic implants, such as the base portion 121 associated with the tibial implant system 120. According to one embodiment, a virtual implant model may be provided by the manufacturer of the prosthetic implant and the graphical user interface 400 may provide a graphical representation of the geometry of the prosthetic implant. Using the graphical representation of the geometry, a virtual haptic boundary (e.g., standard haptic boundary 12, as described below) may be created and associated with the virtual implant model.

The graphical user interface 400 may include a plurality of sub-screens, each of which is configured to display a particular feature of the implant planning phase. For example, the graphical user interface 400 may include a first sub-screen (e.g., upper left) for displaying a virtual implant model (e.g., a virtual model of the base portion 121). The graphical user interface 400 may also include a second sub-screen (upper right) for displaying a virtual bone model associated with the patient's anatomy (e.g., a virtual model of the native tibia 101) upon which the implant (e.g., the base portion 121) will be positioned. The graphical user interface 400 may include a third sub-screen (lower left) for displaying a planned placement of the virtual implant model (e.g., displayed in the first sub-screen) within the patient's anatomy (e.g., displayed in the second sub-screen). The graphical user interface 400 may also include a fourth sub-screen (lower right) for displaying a view of respective medial and lateral resection portions 401a, 401b associated with the planned implant placement. It is contemplated that the number and view of sub-screens may differ from those provided in the exemplary embodiment illustrated in FIG. 4. It is also contemplated that one or more of the sub-screens allow a user to interactively update the view and/or the components within the view. For example, although the lower right screen shows a top view of a simulated resection of the native tibia 101 based on the planned implant placement shown in the lower left sub-screen, it is contemplated that the user can select different views (e.g., front, back, side, bottom, etc.) for displaying the contents of the sub-screen.

During the implant planning stage, a surgeon or medical professional may use planning software associated with the surgical system 200 to plan the placement of prosthetic implants onto or within a patient's anatomy. As such, virtual (i.e., software) 3-D models of prosthetic implants, the patient's anatomy, a surgical instrument (such as the surgical tool 234), and any other physical object that may be used during the surgical procedure may be generated and registered to a virtual coordinate space (generally one that corresponds with the patient's anatomy). Using planning software, the surgeon can virtually position a prosthetic implant relative to the patient's anatomy.

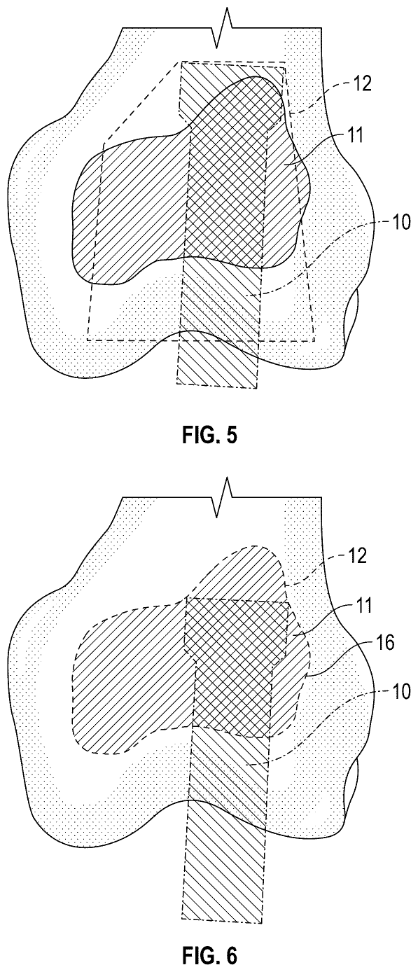

Referring to FIG. 5, the computing system 224 may generate a standard haptic boundary 12 based on the virtual implant model. The standard haptic boundary 12 may be configured to accommodate the cutting profile and size of a cutting tool (e.g., the surgical tool 234), shown schematically as a cutting tool 10. However, as shown, the standard haptic boundary 12 based on the virtual implant model may under-resect and/or over-resect an actual bone area 11 necessary for resection to receive the implant. Over-resection risks damaging soft tissue, such as collateral ligaments in the knee joint, while under-resecting may leave unresected bone that might require snapping or cracking off and/or manual trimming off with a rongeur.

FIG. 6 shows the standard haptic boundary 12 tied to a perimeter 16. The perimeter 16 represents an intersection of a reference plane of the virtual implant model on the actual bone area 11. The standard haptic boundary 12 as shown in FIG. 6, however, may be unable to accommodate a size and shape of the cutting tool 10 in some areas. For example, a cutting tool such as an oscillating saw blade has a wide effective shape, therefore likely cannot fit into the irregular geometry of the haptic boundary 12 shown in FIG. 6 (e.g., confined by the perimeter 16).

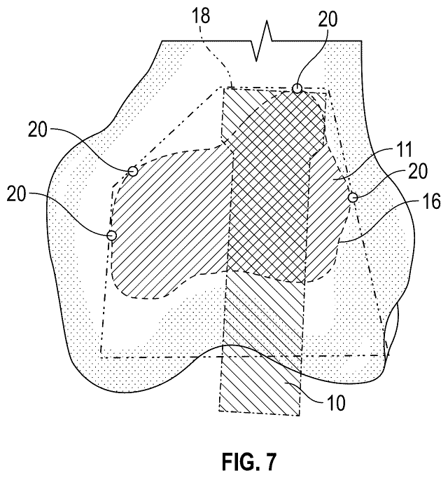

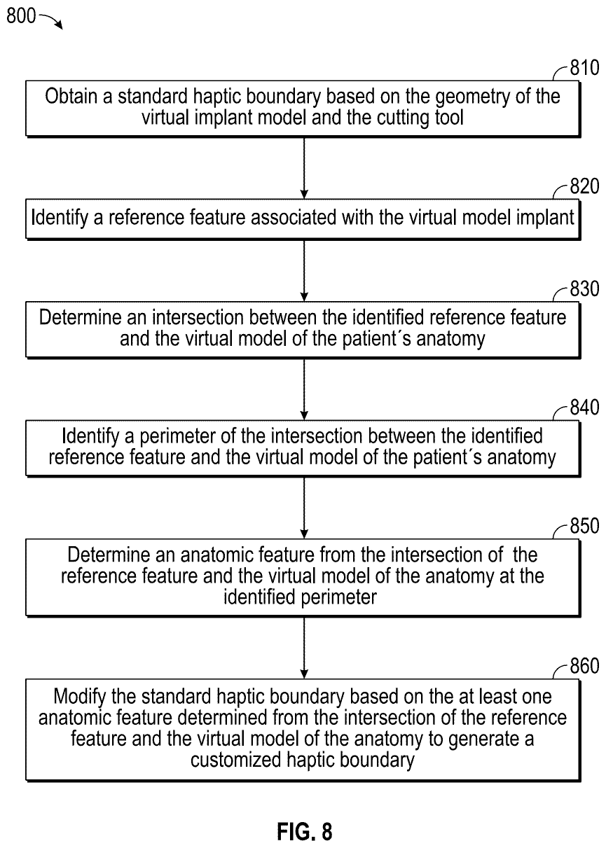

Referring to FIG. 7, a customized haptic boundary 18 based on the configuration of the standard haptic boundary 12 is shown. Unlike the standard haptic boundary 12, however, the customized haptic boundary 18 accommodates for anatomic features 20 positioned along the perimeter 16. The customized haptic boundary 18 can therefore accommodate the cutting tool 10, and more closely matches the necessary areas for bone removal (e.g., the actual bone area 11). In this way, the customized haptic boundary 18 may be configured to minimize under-resecting and/or over-resecting the bone. The generation of the customized haptic boundary 18 is described in detail with respect to the descriptions of FIGS. 8-17 below.

Referring now to FIG. 8, a flowchart illustrating a method 800 for automatically generating a patient-specific virtual haptic boundary (e.g., the customized haptic boundary 18) is shown. According to one embodiment, the method 800 may be implemented during an implant placement planning stage associated with the performance of a surgical procedure (e.g., a TKA procedure). The planning stage may be performed pre-operatively by a surgeon or other medical professional prior to commencement of the surgical procedure. Alternatively or additionally, the planning stage may be performed (or repeated) intra-operatively (e.g., during the surgical procedure).

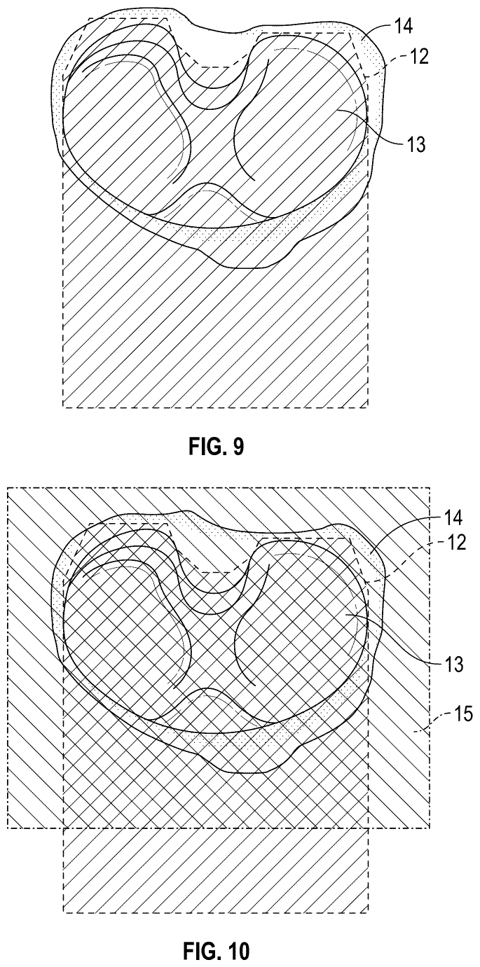

As shown in FIG. 8, once a virtual implant model is placed in a desired position relative to a patient's anatomy, the method 800 commences by identifying the standard haptic boundary 12 based on the size and shape of the virtual implant model (e.g., virtual implant model 13, as shown in FIGS. 9-13) associated with the implant being used, the shape of the cutting tool 10, and the approach angle of the cutting tool 10 (step 810). As such, the standard haptic boundary 12 corresponds closely with the geometric shape of the implant. According to one embodiment, however, the standard haptic boundary 12 may differ from the geometry of the implant. For example, the standard haptic boundary 12 may be slightly larger than the implant to allow sufficient space for the surgical tool 234 (e.g., to accommodate for a width of the cutting tool 10) and/or to provide an area for entering the volume defined by the virtual haptic boundary. The standard haptic boundary 12 is preferably a pre-determined haptic (e.g., from the database 255) tied to the virtual implant model, thus requiring no new haptic generation at step 810. FIG. 9 depicts the standard haptic boundary 12 in relation to the virtual implant model 13 and a virtual model of the patient's anatomy, shown as virtual bone model 14, of the proximal end of a tibia (e.g., the native tibia 101, the tibia 208).

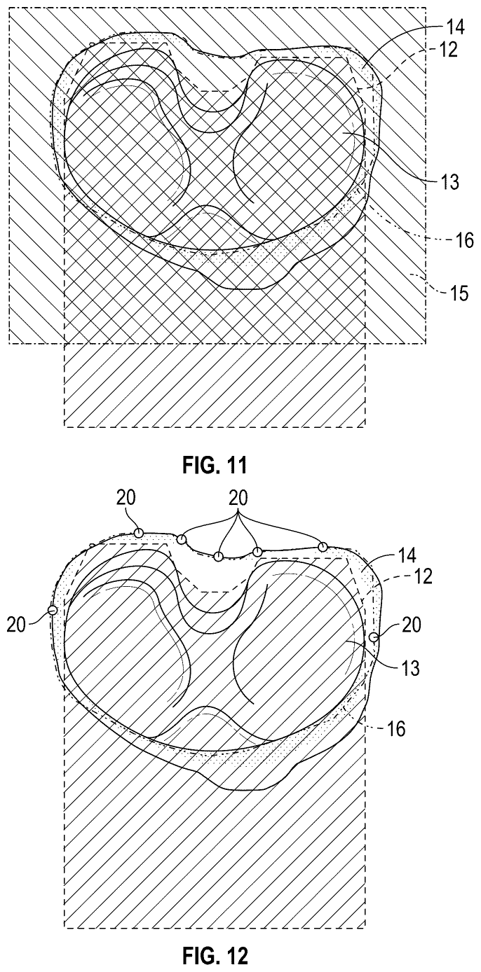

Once the standard haptic boundary 12 is identified at step 810, the position and orientation of a reference feature (e.g., reference feature 15, as shown in FIGS. 10-11) associated with the virtual implant model 13 in a virtual coordinate space is identified (step 820). The reference feature 15 of the virtual implant model 13 may embody one or more points, lines, planes, or surfaces of the virtual implant model 13 and, by extension, the implant associated therewith. The reference feature 15 may refer to a top, a bottom, or other surface of the implant model, or a plane that is otherwise associated with the implant model. In the embodiment shown in FIG. 10, for instance, the reference feature 15 is a plane shown relative to the virtual bone model 14. Alternatively or additionally, the reference feature 15 may include or embody any feature associated with the implant that the surgeon wishes to use as a reference with which to customize virtual haptic boundaries.

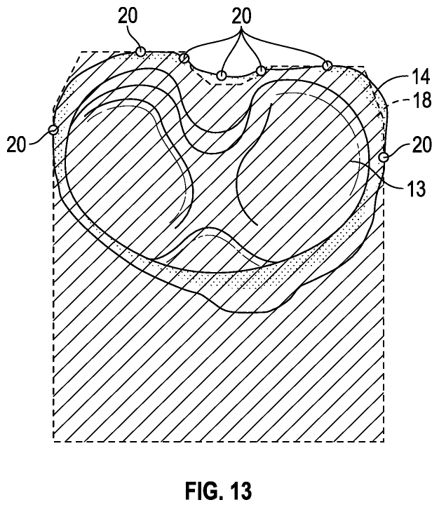

Once the reference feature 15 associated with the virtual implant model 13 has been established at step 820, an intersection between the identified reference feature 15 and the virtual bone model 14 may be determined (step 830). The intersection between the reference feature 15 and the virtual bone model 14 determined at step 830 may then be used to identify a patient-specific anatomic perimeter (step 840). For instance, the patient-specific anatomic perimeter is shown as the perimeter 16 in FIGS. 11-12.

Upon determining the patient-specific anatomic perimeter at step 840, certain features that are specific to the patient's anatomy (e.g., the anatomic features 20) may be identified (step 850). As shown in FIG. 12, for example, the anatomic features 20 are identified along the perimeter 16. The anatomic features 20 may include, for example, a most medial landmark, a most posterior-medial landmark, a most posterior-lateral landmark, and/or a most lateral landmark.

As an alternative or in addition to automatic detection, information indicative of the anatomic features 20, including a modification to the identified anatomic features 20, may be received as a user input. For example, a surgeon may designate one or more points, lines, or areas of the patient's anatomy as anatomic landmarks by physically touching the points, lines, or areas of the patient's anatomy using a probe tool (e.g., the surgical tool 234) that has been registered with the virtual coordinate space. According to another embodiment, a user of the surgical system 200 may input information associated with anatomic landmarks using a graphical user interface associated with the computing system 224. Specifically, the user may select, via the graphical user interface, one or more points, lines, surfaces, or areas on the virtual bone model 14, or along the perimeter 16, using a mouse or other input device. For example, protruding osteophytes on the patient's anatomy may unintentionally create computer-generated landmarks outside of the desired resection region. The surgeon could then deselect such landmarks using the surgical tool 234 or by deselecting the landmark on the virtual bone model 14 using the graphical user interface.

Once the anatomic features 20 are identified on the virtual bone model 14 and/or along the perimeter 16 at step 850, the standard haptic boundary 12 may be modified based on the anatomic features 20 to generate the customized haptic boundary 18 (step 860), as depicted in FIG. 13. That is, the standard haptic boundary 12 may be stretched or shrunk to more closely match the perimeter 16. In certain embodiments, the edges of the standard haptic boundary 12 can be moved based on simple formula percentages. Furthermore, in some embodiments, a particular geometry of the standard haptic boundary 12 can be locked when generating the customized haptic boundary 18 if necessary to prevent disfiguration of the implant shape. In other exemplary embodiments, the standard haptic boundary 12 may be composed of a series of lines or edges made up of vertexes. Each vertex may be designated to stretch in only certain directions, to not stretch (i.e. remain fixed), or to not stretch more than a specified amount. For example, the medial edge vertexes of the standard haptic boundary 12 for the tibial implant system 120 can be designated to only move/stretch medial-lateral and to move/stretch together as a group, so as to maintain the basic shape of the tibial implant system 120. Such control of the vertexes is configured to ensure that the customized haptic boundary 18, once generated from the standard haptic boundary 12, maintains a shape that can accommodate the cutting tool 10.

Alternatively or additionally, step 860 (e.g., modifying the standard haptic boundary 12) may be performed as a manual process by a surgeon. For example, after the perimeter 16 has been identified and/or the anatomic features 20 have been determined (either automatically by the computing system 224 or manually by the surgeon), the surgeon may modify the standard haptic boundary 12 that was previously established or the automatically generated customized haptic boundary 18. In particular, a surgeon may input information regarding modifying the standard haptic boundary 12 using a graphical user interface associated with computing system 224. For example, a surgeon may wish to contract the inner edges of a haptic boundary associated with the base portion 121 of the tibial implant system 120 to limit the operation of the cutting tool near the tibial eminence 101a and avoid the possibility of inadvertently damaging soft tissues (e.g., ACL 103a, PCL 103b) that attach thereto. To do so, the surgeon may select, via a graphical user interface, one or more boundaries or vertexes of the haptic boundary and apply a manipulation to stretch/move the boundary, using a mouse or other input device.

In one embodiment, the surgeon sets a series of offset preferences for the stretchable boundaries. For example, the surgeon may desire that the haptic boundary be offset outwardly from an anatomic landmark by a set distance to enable the cutting tool to cut outside the bone perimeter for improved cutting efficiency. Conversely, the surgeon may desire to set the haptic boundary offset inwardly from an anatomic landmark by a set distance to conservatively protect soft tissues.

Once generated, the customized haptic boundary 18 may be registered to the patient's anatomy and displayed on a display (e.g., display 264) of the surgical system 200. Specifically, when the customized haptic boundary 18 is generated, the computing system 224 may be configured to map the virtual surfaces and features that define the customized haptic boundary 18 to the virtual coordinate space associated with the patient's anatomy. As such, the boundary surfaces associated with the customized haptic boundary 18 are linked to the patient's anatomy, thereby defining the areas of the patient's anatomy within which the surgical instrument is permitted to operate. By registering the customized haptic boundary 18 to the patient's anatomy, the customized virtual haptic boundary 18 becomes virtually linked to the patient's anatomy. Furthermore, in this way, the customized haptic boundary 18 can be tracked (and viewed) relative to movements, modifications, and adjustments in the patient's anatomy during the surgical procedure. The surgical system 200 may then apply the customized haptic boundary 18 to a surgical instrument (e.g., the surgical tool 234).

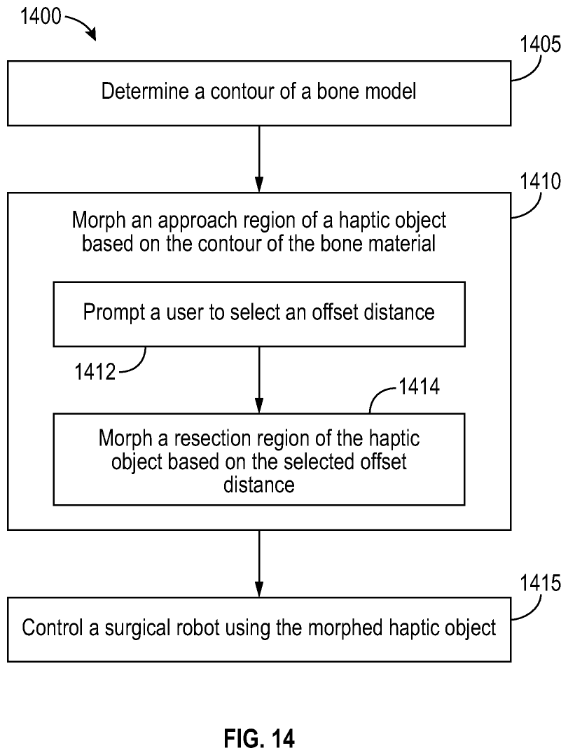

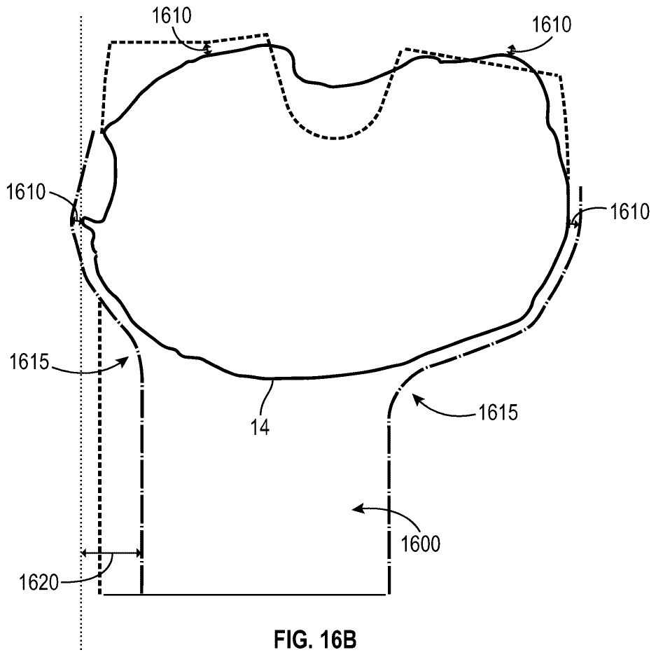

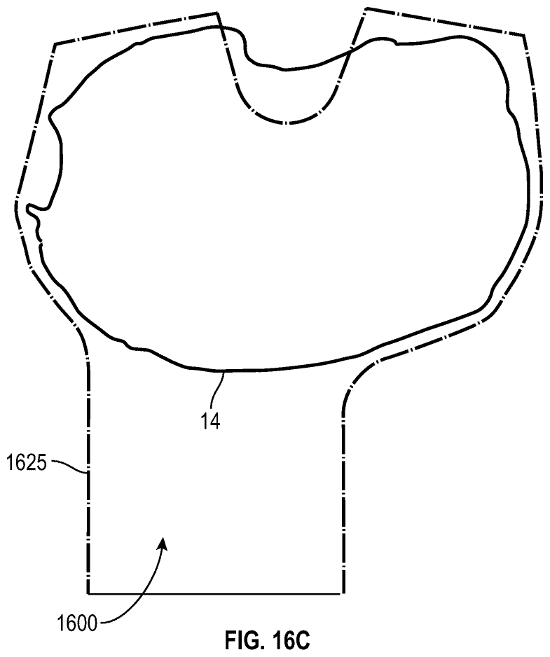

In some embodiments, and as described below with reference to FIGS. 14-16C, generating the customized haptic boundary 18 may include generating a customized haptic approach region. In the examples herein, the haptic object includes a resection region intersecting the bone model and an approach region (e.g., haptic approach region 1600) extending away from (not intersecting) the resection region, as shown in FIGS. 16A-16C. The approach region refers to a portion of the haptic object by which the surgical tool 234 reaches the resection region from a starting position away from the bone, away from the patient, outside the surgical field, etc., while the resection region refers to the actual bone area 11 for resection (e.g., an identified bone area necessary for resection to receive the planned implant in a planned pose). As will be described in detail below, a standard and/or customized haptic boundary can be modified to provide a customized (patient-specific) approach region such that the haptic boundary is configured to constrain a cutting tool within an a customized area while approaching a planned resection region (e.g., before reaching the bone, to protect soft tissue in a patient-specific manner).

Referring to FIG. 14, a method 1400 for generating a customized haptic approach region (e.g., morphed haptic object 1625, as shown in FIG. 16C) is shown. The method 1400 can be executed automatically by the surgical system 200, for example by the computing system 224 and/or a combination of computing components (e.g., at least in part by a separate planning computing system, such as a cloud-based platform).

As shown, the method 1400 begins by determining a contour of a bone model (step 1405). In some embodiments, the bone model refers to the virtual bone model 14. That is, the bone model represents a bone (e.g., the tibia 208) being operated on during a surgical procedure (e.g., a TKA procedure). Furthermore, according to some embodiments, the bone model is a three-dimensional (3D) mesh. The contour of the bone model is determined at a cross-section intersected by a haptic object (e.g., the standard haptic boundary 12, the customized haptic boundary 18, a planar haptic object) corresponding to a planned resection (e.g., a planned resection during a TKA procedure, as described herein). As described herein, the planned resection may be planned based on a geometry of an implant (e.g., a size of the implant, a type of implant, etc.) being implanted in the resection region. Thus, the resection region may be determined based on the geometry of the implant.

After determining the contour of the bone model at step 1405, the method 1400 includes morphing the approach region of a haptic object based on the contour of the bone model (step 1410). In some embodiments, the haptic object refers to the standard haptic boundary 12 and/or the customized haptic object 18. Where the haptic object refers to the standard haptic boundary 12, step 1410 may further include generating the customized haptic object 18 from the standard haptic boundary 12, as described above. In other words, generating the customized haptic approach region as described herein also includes modifying the standard haptic boundary 12 based on the patient-specific anatomy. The modification to the standard haptic boundary 12 (e.g., as described by the method 800) may be performed prior to the method 1400 or concurrently with the method 1400. That is, where the modification to the standard haptic boundary 12 is performed prior to the method 1400, the haptic object referred to by method 1400 may refer to the customized haptic object 18. On the other hand, where the modification to the standard haptic boundary 12 is performed concurrently with the method 1400, the haptic object referred to by method the 1400 may refer to the standard haptic object 12. FIGS. 16A-16C, for instance, depict an implementation of the method 1400 in which the modification to the standard haptic boundary 12 is performed concurrently with the generation of the customized haptic approach region, and thus the haptic object refers to the standard haptic boundary 12.

In some embodiments, morphing the approach region at step 1410 includes narrowing the approach region by curving at least a portion of the haptic object to follow an anterior portion of the contour of the bone model. For example, the anterior portion of the contour of the bone model is depicted at radius 1615 in FIG. 16B and described in greater detail below. Additionally or alternatively, morphing the approach region at step 1410 may include narrowing the approach region by or to a predetermined distance (e.g., described during step 1515 of method 500 and shown as distance 1620 in FIG. 16B). The predetermined distance refers to an input parameter configured to determine a width of the morphed approach region. In some embodiments, the width of the morphed approach region may depend on a width of the surgical tool 234 being used to perform the TKA procedure. As shown in FIGS. 16A-16C, morphing the approach region may include adjusting at least one of a posterior segment, a medial side, or a lateral side of the haptic object.

In some instances, morphing the approach region of the haptic object at step 1410 includes prompting a user (e.g., a surgeon) to select an offset distance (step 1412). The offset distance selected by the user may include the series of offset preferences of the surgeon, as described above. For example, the user may select an offset distance configured to offset the haptic object outwardly from an anatomic landmark (e.g., the anatomic features 20) by a set distance (e.g., such that an extended boundary is provided which can increase cutting efficiency by allowing tool movement through more space including outside the bone perimeter). As another example, the user may select an offset distance configured to offset the haptic object inwardly from an anatomic landmark (e.g., the anatomic features 20) by a set distance (e.g., to conservatively protect soft tissues from the cutting tool). The offset distance is shown as offset distance 1610 in FIGS. 16A-16C.

Based on the selected offset distance received in response to the prompt at step 1412, step 1410 may further include morphing the resection region of the haptic object based on the selected offset distance (step 1414). In other words, and as shown in FIGS. 16A-16C, the resection region may be morphed by shifting edges of the haptic object to positions offset from the contour of the bone model by the selected offset distance.

After morphing the approach region at step 1410, a surgical robot (e.g., the robotic device 220) is controlled using the morphed haptic object (step 1415), for example such that the surgical robot constrains a cutting tool to the morphed haptic object, providing haptic feedback, force feedback, etc. as described elsewhere herein. Furthermore, the surgical robot interfaces with a cutting tool (e.g., the surgical tool 234) that reaches the resection region via the approach region. In some embodiments, controlling the surgical robot at step 1415 includes guiding the cutting tool to perform a tibial resection during a TKA procedure. Step 1415 may also include displaying (e.g., via the display 264) a virtual model of the bone model and an outline of the morphed haptic object overlaid on the virtual model. In some embodiments, at step 1415, the surgical robot is controlled to automatically move the cutting tool through the morphed haptic object to provide automated bone resection using the morphed haptic object.

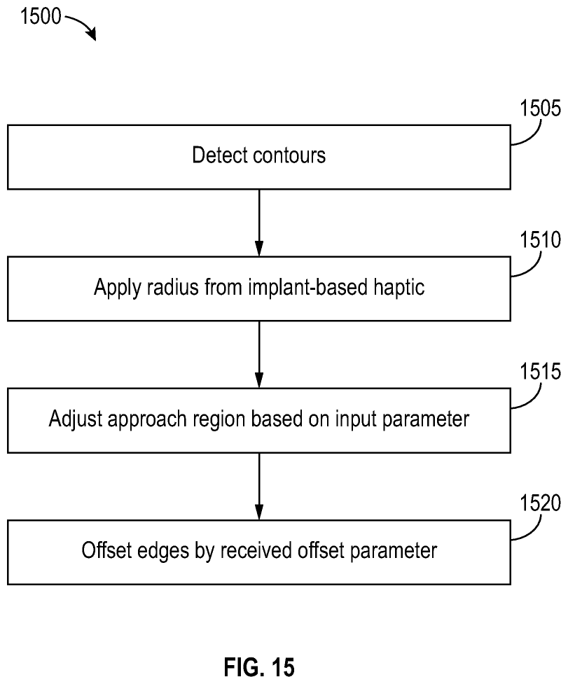

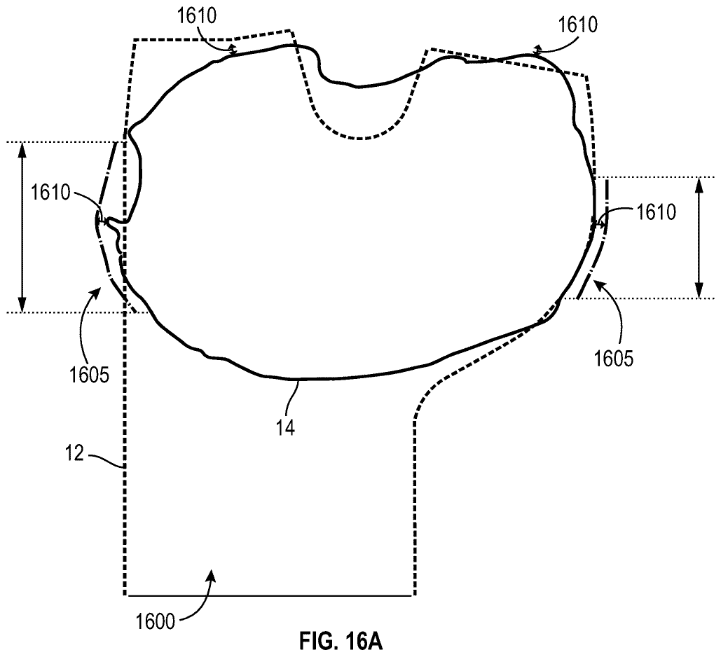

Referring now to FIG. 15, a method 1500 for calculating the customized haptic approach region during the method 1400 is shown. The method 1500 can be executed automatically by the surgical system 200, for example by the computing system 224 and/or a combination of computing components (e.g., at least in part by a separate planning computing system, such as cloud-based platform). As described above with reference to step 1405 of method 1400, calculating the customized haptic approach region may begin by detecting contours (step 1505). For example, FIG. 16A depicts contours 1605 of the virtual bone model 14. The contours 1605 are depicted to be offset from the virtual bone model 14 by an offset distance 1610 (e.g., the offset distance selected by the user at step 1412 of method 1400). In some embodiments, the contours 1605 are detected automatically by fitting a curve to points along medial and/or lateral side(s) of the virtual bone model 14 at a depth along a length of the bone determined based on planned implant placement (e.g., at an intersection of the virtual bone model 14 and a planned bone surface to be provided as a result of a bone resection to prepare the bone to receive the implant in a planned position and orientation) and/or otherwise estimated a contour of one or more portions of an exterior surface of the bone model. The contour can follow the exterior of the virtual bone model 14 including at least partially around a anterior portion (or other surgical approach side) of the virtual bone model 14. As illustrated in FIGS. 16A-B, determining a contour can include smoothing over or otherwise accounting for osteophytes, imaging artifacts, or other irregularities, for example so as to identify a contour with at least a minimum radius of curvature.

Once the contours are detected at step 1505, the method 1500 includes generating a transition between the resection region and the approach region of the haptic object by applying a radius from an implant-based haptic (step 1510). For example, an implant-based haptic boundary can be stored with a radius value to be applied in step 1510 for morphing an approach region of the haptic boundary. The implant-based haptic may refer to the standard haptic boundary 12. For instance, FIG. 16B depicts a radius 1615 which is applied to morph the haptic boundary. That is, referring to FIGS. 16A-B, the radius 1615 is applied to create a curvature between the contour 1605 to a substantially straight approach boundary extending away from the virtual bone model 14. Accordingly, the radius 1615 is applied to taper the haptic approach region 1600 along an anterior portion of the virtual bone model 14, i.e., a direction from which a cutting tool will approach the bone to initiate a bone resection, while providing a smooth transition to the approach region. As shown in FIG. 16B, the radius 1615 is applied to both medial and lateral sides of the haptic approach region 1600 at step 1505. In some embodiments, the contour 105 and the radius 1615 are used together with a target width for the surgical approach region 1600 to create a continuous haptic boundary tapered around at least a portion of the anterior portion of the virtual bone model 14.

Method 1500 continues with adjusting the haptic approach region 1600 based on an input parameter (step 1515), for example received from a user via a graphical user interface or determine based on one or more tracked probe positions. The input parameter refers to the distance (e.g., shown as distance 1620 in FIG. 16B) by which the approach region is narrowed at step 1410 and is depicted in FIG. 16B as distance 1620. In the example shown, an edge of the approach region of a standard haptic boundary 12 is shifted inwardly from a maximum width of the resection region by the distance 1620 to narrow the haptic approach region 1600. Such an adjustment can protect soft tissue neighboring the approach region. Enabling user adjustment of the distance 1620 can allow the user to customize the size of the approach region 1600 in accordance with user preferences for ease of access to resection region relative to desire for increase constraint and guidance for approaching the resection region.

In some embodiments, the distance 1620 and/or other spacing or positioning for customizing the approach region 1600 is based on one or more tracked probe positions. For example, a probe trackable by the tracking system 222 of the surgical system 200 can be touched to a bone at medial and/or lateral borders of an approach area and such probe positions can be used to define the distance 1620 or other parameter or positioning for adjustment of the approach area. In such embodiments, a surgeon can physically move the probe to one or more positions near soft tissue (e.g., adjacent to the patellar tendon) and hold the probe to such position(s) while the probe position relative to the bone is captured by the tracking system 222. The probe positions can then be used in morphing a haptic object, for example by narrowing the approach region 1600 by a sufficient amount for the tracked probe positions to fall outside the morphed haptic object 1625.

As shown in FIG. 15, morphing the haptic object can also include offsetting edges of the haptic object from the bone model 14 by the offset parameter received during method 1400, as described above (1520), including at posterior edges of the haptic object and medial and lateral sides of the bone model 14 (see distance 1610 in FIG. 16B). Completion of method 1500 may therefore result in generation of the morphed haptic object 1625, as shown in FIG. 16C, which follows contours of the bone model 14 including around a portion of an anterior side of the bone model 14 so as to constrain the space through which a cutting tool can reach the actual bone represented by the bone model 14.

In some embodiments, the morphed haptic object 1625 may be used in place of the customized haptic object 18 in functions and features described elsewhere herein (e.g., step 1750 below), while providing a patient-specific, customized approach region (e.g., a customization of the haptic approach region 1600).

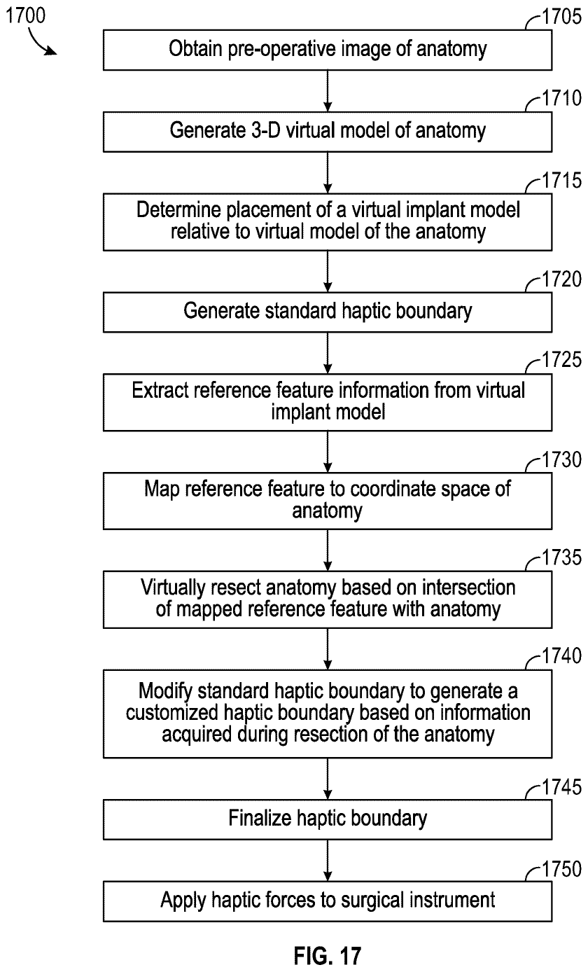

FIG. 17 shows a flowchart of another exemplary method 1700 for customizing a haptic boundary based on patient-specific parameters. As illustrated in FIG. 17, method 1700 commences upon receipt of pre-operative image(s) or image data associated with a patient's anatomy (step 1705). Pre-operative image(s) may include any two-or three-dimensional image data set obtained using any suitable imaging process for recording images associated with a patient's anatomy such as, for example, x-ray, computed tomography (CT), magnetic resonance (MR), positron emission tomography (PET), single photon emission computed tomography (SPECT), ultrasound, etc. According to one embodiment, the I/O devices 262 of the surgical system 200 may receive pre-operative CT scan data 258 associated with the anatomy of the specific patient that is to be operated on.

Upon receiving the pre-operative image(s) of the anatomy of the patient, the computing system 224 generates a 3-D virtual model of the patient's anatomy (step 1710). That is, the 3-D virtual model of the patient's anatomy may refer to the virtual bone model 14. In some embodiments, computing system 224 may include one of a number of different software tools for rendering 3-D models of objects, based on the received 2-D (or 3-D) image data sets associated with the anatomy of the patient. In an alternative embodiment, the 3-D virtual model of the patient's anatomy is generated utilizing an imageless system.

After the virtual bone model 14 of the patient's anatomy is generated, it may be registered with the actual anatomy of the patient so that surgical system 200 can virtually track (e.g., using the tracking system 222, as described above) the position and orientation of the actual anatomy of the patient in virtual software space. According to one embodiment, this registration process involves associating a plurality of points of the patient's anatomy with corresponding points on the virtual bone model 14. Such associations can be made using a probe tool that has been registered in the virtual coordinate space, whereby a plurality of points on the patient's anatomy are gathered by touching or “exploring” one or more surfaces of the patient's anatomy using a tip of the probe tool. Once the virtual bone model 14 is registered with the patient's anatomy, the tracking system 222 may be configured to track the position and orientation of the patient's anatomy in the virtual coordinate space.

After the virtual bone model 14 is generated and registered to the patient's bone, the computing system 224 may facilitate the planning of a prosthetic implant within the patient's anatomy (step 1715). Specifically, the computing system 224 determines, based on a user input, placement of a virtual implant model (e.g., the virtual implant model 13) relative to the virtual bone model 14. For example, a surgeon may select the virtual implant model 13 (e.g., the virtual model associated with base portion 121, as shown in FIG. 4) from a database (e.g., the database 255) of implants available for the surgery. Then, using a graphical user interface (e.g., the graphical user interface 400), the surgeon may manipulate the position of the virtual implant model 13 relative to the patient's anatomy (e.g., the native tibia 101), which produces a virtual representation of the patient's anatomy fitted with the virtual implant model 13 (e.g., as shown in the lower left sub-screen of the graphical user interface 400 shown in FIG. 4). Such a process for virtually planning implant placement allows the surgeon to make precise adjustments to the position of the implant relative to the patient's anatomy in a simulated software environment prior to commencing the bone resection process.

Once the placement of the virtual implant model 13 with respect to the virtual bone model 14 is finalized, the standard haptic boundary 12 is generated (step 1720). The standard haptic boundary 12 may correspond closely with the geometric shape of the prosthetic implant. Then, reference feature information (e.g., relating to the reference feature 15) is extracted from the virtual implant model 13 (step 1725). According to one embodiment, the reference feature 15 of the virtual implant model 13 may embody one or more points, lines, planes, or surfaces of the virtual implant model 13. As described above and shown in FIG. S10-11, the reference feature 15 may be a plane associated with the implant model 13. Alternatively or additionally, the reference feature 15 may include or embody any feature associated with the implant that the surgeon wishes to use as the reference with which to customize virtual haptic boundaries. For example, the reference feature 15 may include any surface associated with the virtual implant model 13 that directly abuts or faces a surface of the virtual bone model 14.

Upon extracting the reference feature information, the computing system 224 maps the reference feature information onto the coordinate space of the patient's anatomy (step 1730). That is, the computing system 224 is configured to register the reference feature 15 of the virtual implant model 13 to the virtual bone model 14, such that the reference feature 15 is tracked relative to the position of the patient's bone.

Method 1700 may also include the computing system 224 determining an intersection between the reference feature 15 (e.g., once the reference feature 15 is mapped onto the coordinate space of the patient's anatomy) and the virtual bone model 14, and virtually resecting tissue based on the determined intersection (step 1735). The computing system 224 may also be configured to modify the standard haptic boundary 12 to generate the customized haptic boundary 18 (step 1740) based on information acquired during resection of the anatomy. As described above, the customized haptic boundary 18 may be generated by stretching/moving the standard haptic boundary 12 based upon at least one anatomic feature 20 located along the perimeter 16, which is defined by the intersection of the reference feature 15 and the virtual bone model 14. As described above, the identification of the anatomic features 20 may be done automatically by the surgical system 200 or may be performed manually by the surgeon. Similarly, the modification of the standard haptic boundary 12 may be performed automatically based on the previously identified anatomic features 20, or may be performed manually by the surgeon who moves/stretches the standard haptic boundary 12 based on the patient specific anatomy or according to desired cutting approaches and techniques. For example, the surgeon may manually move/stretch the standard haptic boundary 12 to limit the operation of the cutting tool near the tibial eminence 101a and to avoid the possibility of inadvertently damaging soft tissues (e.g., ACL 103a, PCL 103b) that attach thereto.

Upon generating the customized virtual haptic boundary 18 (or the morphed haptic object 1625), the surgical system 200 provides the user with an option to finalize the virtual haptic boundary (step 1745). When the user decides to finalize the virtual haptic boundary, the surgical system 200 may update the force system with the coordinates of virtual haptic boundary. As such, the surgical system 200 selectively applies the virtual haptic forces to the surgical tool 234 based on the tracked position of the surgical tool 234 relative to the virtual haptic boundary (step 1750).

The presently disclosed systems and methods for customizing virtual haptic boundaries provide a solution for adjusting virtual haptic boundaries associated with force feedback control system for computer-assisted surgery systems. According to one embodiment, this solution allows a user to modify a haptic boundary by stretching or contracting an existing haptic boundary to fit one or more anatomic landmarks. The planning software may then determine an intersection between the stretched (or contracted) boundary and the virtual model of the patient's anatomy to define the location of the new virtual haptic boundary, and establish the new virtual haptic boundary based on the determined intersection.

In some embodiments, data is collected relating to the planning and procedures conducted using the systems and methods described herein. For example, details such as the types of implants used, bone density, ligament balancing measurements, final implant placement (angle, anterior/posterior placement, medial/lateral placement, placement with respect to a joint line, mechanical and anatomic axis positions, etc.), among other possibilities, can be collected during planning of the procedures. Post-operative outcomes may also be collected. The post-operative outcomes may then be compared to the other data to provide insights into improved execution and implementation of the systems and methods described herein.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and are considered to be within the scope of the disclosure.

What is claimed is:

1. A method, comprising:
determining a contour of a bone model at a cross-section intersected by a haptic object corresponding to a planned resection, the haptic object comprising a resection region intersecting the bone model and an approach region extending away from the resection region;
morphing the approach region of the haptic object based on the contour of the bone model; and
controlling a surgical robot using the morphed haptic object, wherein the surgical robot interfaces with a cutting tool that reaches the resection region via the approach region.

2. The method of claim 1, wherein morphing the approach region comprises narrowing the approach region by curving at least a portion of the haptic object to follow an anterior portion of the contour of the bone model.

3. The method of claim 1, further comprising prompting a user to select an offset distance and morphing the resection region by shifting edges of the haptic object to positions offset from the contour of the bone model by the selected offset distance.

4. The method of claim 1, wherein the planned resection is planned based on a geometry of an implant being implanted in the resection region, wherein the method further comprises morphing the resection region of the haptic object based on intersection of the bone model and the haptic object.

5. The method of claim 1, further comprising tracking a probe position as the probe is touched to a bone or to soft tissue proximate the bone, wherein morphing the approach region is further based on the tracked probe position.

6. The method of claim 1, further comprising displaying a virtual model of the bone model and an outline of the morphed haptic object overlaid on the virtual model.

7. The method of claim 1, wherein the bone model is a three-dimensional (3D) mesh.

8. The method of claim 1, wherein controlling the surgical robot comprises guiding the cutting tool to perform a tibial resection of a total knee arthroplasty (TKA) procedure.

9. The method of claim 1, wherein morphing the approach region is configured to adjust at least one of a posterior segment, a medial side, or a lateral side of the haptic object.

10. A system comprising:
a robotic device; and
circuitry programmed to:
determine a contour of a bone model at a cross-section intersected by a haptic object corresponding to a planned resection, the haptic object comprising a resection region intersecting the bone model and an approach region extending away from the resection region;
morph the approach region of the haptic object based on the contour of the bone model; and
control the robotic device using the morphed haptic object, wherein the surgical robot interfaces with a cutting tool that reaches the resection region via the approach region.

11. The system of claim 10, wherein morphing the approach region comprises narrowing the approach region by curving at least a portion of the haptic object to follow an anterior portion of the contour of the bone model.

12. The system of claim 10, further comprising prompting a user to select an offset distance and morphing the resection region by shifting edges of the haptic object to positions offset from the contour of the bone model by the selected offset distance.

13. The system of claim 10, wherein the planned resection is planned based on a geometry of an implant being implanted in the resection region, wherein the circuitry is further programmed to morph the resection region based on the bone model.

14. The system of claim 13, further comprising a tracking system configured to track a position of a probe, wherein the circuitry is programmed to morph the approach region further based on the position of the probe.

15. The system of claim 10, further comprising displaying a virtual model of the bone model and an outline of the morphed haptic object overlaid on the virtual model.

16. The system of claim 10, wherein the bone model is a three-dimensional (3D) mesh.

17. The system of claim 10, wherein controlling the robotic device comprises guiding the cutting tool to perform a tibial resection of a total knee arthroplasty (TKA) procedure.

18. The system of claim 10, wherein morphing the approach region is configured to adjust at least one of a posterior segment, a medial side, or a lateral side of the haptic object.

19. One or more non-transitory computer-readable media storing instructions that, when executed by a processor, cause the processor to perform operations comprising:
determining a contour of a bone model at a cross-section intersected by a haptic object corresponding to a planned resection, the haptic object comprising a resection region intersecting the bone model and an approach region extending away from the resection region;
morphing the approach region of the haptic object based on the contour of the bone model; and
controlling a surgical robot using the morphed haptic object, wherein the surgical robot interfaces with a cutting tool that reaches the resection region via the approach region.

20. The one or more non-transitory computer-readable media of claim 19, wherein morphing the approach region comprises narrowing the approach region by curving at least a portion of the haptic object to follow an anterior portion of the contour of the bone model.
